(19) United States
(12) Patent Application Publication
(10) Pub. No.: US 2025/0295664 A1
(43) Pub. Date: Sep. 25, 2025

(54) THERAPEUTIC COMBINATION OF KRAS G12C INHIBITOR AND TEAD INHIBITOR

(71) Applicant: SANOFI, PARIS (FR)

(72) Inventors: Jean-Christophe LE BAIL, Paris (FR); Jurgen MOLL, Paris (FR); Salvina TAMMACCARO, Paris (FR); Iris VALTINGOJER, Paris (FR)

(21) Appl. No.: 18/852,884

(22) PCT Filed: Apr. 3, 2023
(86) PCT No.: PCT/EP2023/058693

§ 371 Date: Sep. 30, 2024

(30) Foreign Application Priority Data
Apr. 4, 2022 (EP) 22305439.6

(51) Int. Cl. A61K 31/519; A61K 31/167; A61P 35/00

(52) U.S. Cl. CPC A61K 31/519; A61K 31/167; A61P 35/00

(57) ABSTRACT

The invention relates to a therapeutic combination comprising at least one KRAS G12C inhibitor and at least one specific TEAD inhibitor selected from the group consisting of molecules of formula (III), indole compounds of formula (IV), indane compounds of formula (V) and the pharmaceutically acceptable salts thereof; and to its use in the treatment of KRAS G12C-mediated cancer, in particular of lung, pancreatic or colorectal cancer. The present invention also concerns a pharmaceutical composition comprises this therapeutic combination and a kit.

The present disclosure relates to a therapeutic combination of at least one KRAS G12C inhibitor and at least one specific TEAD inhibitor.

The therapeutic combination disclosed herein is particularly advantageous in the treatment of KRAS G12C-mediated cancers, and more particularly in the treatment of lung, pancreatic or colorectal cancer.

RAS proteins, a family of small GTPases that integrate and transmit signals from upstream growth factor receptors, comprise the most frequently mutated protein family in human cancer and high frequency of RAS mutations are found with the top three causes of cancer deaths: lung, colorectal, and pancreatic cancer. RAS proteins function as a molecular switch. In conditions of normal signaling, ligand stimulation results in activation of the guanine nucleotide exchange factor son of sevenless (SOS) and facilitates exchange of the inactive guanosine diphosphate (GDP)-bound state of RAS to an active guanosine triphosphate (GTP)-bound state. This switch between inactive and active states enables RAS to adopt a conformation that interacts with the RAS-binding domain (RBD) of its downstream effectors and facilitates the recruitment of rapidly accelerated fibrosarcoma (RAF) family members (ARAF, BRAF, CRAF) from the cytosol to the plasma membrane which eventually leads to the activation of the MAPK signaling cascade. Active MAPK signaling further results in the activation of gene transcription programs required for cell proliferation. Under normal conditions, the activation of the RAS-RAF-MAPK signaling cascade is transient and is turned off via the action of RAS-GTPase activating (GAP) proteins. These proteins activate GTPase enzymes found within RAS, which hydrolyze GTP to GDP and therefore switch RAS off. Mutations in RAS proteins may lead to conformational changes so that the RAS-GAP protein cannot activate the inherent GTPase enzyme anymore. As a result, the GTP molecules are not hydrolysed and instead they maintain RAS continuously in its active state, thus causing pro tumorigenic effects by amplifying signaling in the MAPK pathway (reviewed in Hymowitz and Malek, CHS Perspectives 2018:8 (11)).

Recently, small molecules specifically targeting the KRAS G12C oncogenic mutant protein have advanced in clinical trials (reviewed in Goebel et al, RSC Medicinal Chem: 7, 2020). The KRAS G12C refers to a mutant form of the mammalian KRAS protein that contains an amino acid substitution of a cysteine for a glycine at amino acid position 12. The assignment of amino acid codon and residue positions for human KRAS is based on the amino acid sequence identified by UniProtKB/Swiss-Prot P01116: Variant p.Gly12Cys.

On the other hand, the transcriptional enhanced associate domain (TEAD) family of transcription factors TEAD1-TEAD4 are the most downstream effectors of the HIPPO-YAP1 signaling cascade, an evolutionary conserved signaling pathway whose deregulation is described for different cancer types (reviewed in Nguyen and Yi, Trends Cancer. 2019, 5:283-296). The core of the HIPPO pathway in mammals consists of a cascade of kinases including MST1/2 and LATS1/2, their associated adaptor proteins SAV1 and MOB1, as well as upstream regulators, such as NF2, SCRIBBLE, CRUMBS, and multiple G protein-coupled receptors. In the healthy adult human organism, the HIPPO pathway kinases are mainly found in their “on” state in which they actively phosphorylate YAP1 and TAZ (WWTR1 gene) proteins. Phosphorylated YAP1 and TAZ then remain inactive through sequestration in the cytoplasm and/or degradation by the proteasomal machinery. In many tumors, HIPPO signaling is found in the “off” state, in which case cytosolic YAP1 and TAZ proteins are not anymore phosphorylated and hence free to translocate to the cell nucleus, where they associate with TEAD transcription factors. The YAP1-TEAD or TAZ-TEAD couples then bind to DNA and induce the expression of genes that promote cell proliferation and cell survival (reviewed in Totaro et al., Nature Cell Bio. 2018, 20:888-899).

Small molecule allosteric ligands binding to the central lipid pocket of TEAD proteins are capable of inhibiting aberrant YAP1-TEAD or TAZ-TEAD activation and several such allosteric TEAD inhibitors have been described in the literature with K-975 being one of the publicly known examples (Kaneda et al., Am J Cancer Res. 2020, 10:4399-4415). Allosteric TEAD inhibitors inhibit the growth of tumor cells in vitro and in vivo and are active in tumor types that depend on TEAD activity and where activation of TEAD (YAP1-TEAD or TAZ-TEAD) is the main driver of tumor growth. This is for example the case in tumor indications with dysfunctional HIPPO signaling, which is for example the case in malignant mesothelioma tumors with HIPPO kinase or NF2 inactivation (Kaneda et al.). In tumors, in which TEAD is not the driver of tumor cell growth, TEAD inhibitors have no effect and can be added to in vitro and in vivo models without any impact on tumor cell proliferation and survival.

As every signaling pathway, the HIPPO-YAP1/TAZ-TEAD signaling cascade does not exist in isolation but cross-talks with other signaling pathways. For example, the crosstalk between this pathway and the MAPK pathway has recently been reported (Pham et al., Cancer Discovery 2020, 11:778-793). The MAPK pathway is tightly regulated by RAS proteins.

The present disclosure provides the unexpected finding that the combination of a specific TEAD inhibitor with a KRAS G12C inhibitor is particularly effective in the treatment of tumors harboring KRAS G12C mutations, and thus for its use in the treatment of KRAS G12C-associated cancers, including lung adenocarcinoma, pancreatic ductal adenocarcinoma, rectum adenocarcinoma, colon adenocarcinoma, bile duct carcinoma, chronic myelomonocytic leukemia (CMML), rhabdomyosarcoma, endometrial cancer, bladder cancer, and ovarian cancer (Li et al., Nat Rev Cancer, 2018 December; 18 (12): 767:777).

Thus, according to one of its aspects, the present disclosure provides a therapeutic combination comprising at least one KRAS G12C inhibitor and at least one specific TEAD inhibitor selected from the group consisting of molecules of formula (III), indole compounds of formula (IV), indane compounds of formula (V) as detailed hereafter and the pharmaceutically acceptable salts thereof.

Surprisingly, as shown in the tests of the examples set out below in different KRAS tumor models with KRAS G12C mutations, the inventors have discovered that the addition of said TEAD inhibitor in combination with a KRAS G12C inhibitor makes it possible to significantly improve the inhibition of the KRAS G12C mutant cell line growth, in comparison with the effect achieved with the KRAS G12C inhibitor alone.

Thus, the presence of said TEAD inhibitor makes it possible to stimulate/potentiate the inhibition effect of the KRAS G12C inhibitor.

As illustrated in the examples, the combination of said TEAD inhibitor with said KRAS G12C inhibitor surprisingly potentiates the effect of the KRAS G12C inhibitor by several orders of magnitude and up to 100× in some cases. This significant potentiation effect for the inhibition of KRAS G12C is especially even more surprisingly, given that these specific TEAD inhibitors alone are totally inactive on tumor models with KRAS G12C mutations.

In an embodiment, the therapeutic combination combines one or more KRAS G12C inhibitors as disclosed hereinafter and one or more TEAD inhibitors as disclosed hereinafter.

As used herein, certain terms have the following definitions:
a halogen atom: a fluorine, a chlorine, a bromine or an iodine atom;
an alkyl group: a linear or branched saturated aliphatic group. More particularly, a (Cx-Cy) alkyl group, where x and y are integers, x<y, is a linear or branched saturated aliphatic group comprising from x to y carbon atoms, for example from 1 to 4 carbon atoms. By way of examples, mention may be made of, but not limited to the groups methyl, ethyl, propyl, isopropyl, and the like;
a cycloalkyl group: a cyclic alkyl group, including spiro groups. More particularly, a (C3-Cz) cycloalkyl group, where z is an integer greater than or equal to 4, is a cyclic alkyl group comprising, unless otherwise mentioned, from 3 to z carbon atoms, and being unsubstituted or substituted. By way of examples, mention may be made of, but not limited to the groups cyclopropyl, cyclobutyl, cyclopentyl, cyclohexyl, spiro[2.3]hexanyl, and the like;
an alkenyl group: a linear or branched mono- or polyunsaturated aliphatic group containing, for example, one or two ethylenic unsaturations. More particularly, a (C2-Cy) alkenyl group, where y is an integer greater than or equal to 3, is a linear or branched mono- or polyunsaturated aliphatic group containing from 2 to y carbon atoms, for example from 2 to 4 carbon atoms, and containing, for example, one or two ethylenic unsaturations. By way of examples, mentioned may be made of, but not limited to ethenyl, propenyl, n-propenyl, isopropenyl, butenyl, isobutenyl, sec-butenyl, tert-butenyl groups, and the like;
an alkoxy group: a radical-O-alkyl in which the alkyl group is as defined above.

More particularly, a (Cx-Cy) alkoxy group, where x and y are integers, x<y, is an —O—(Cx-Cy) alkyl group where the (Cx-Cy) alkyl group is as previously defined. For example, the alkoxy group is a (C1-C4) alkoxy group. By way of examples, mention may be made of, but not limited to methoxy, ethoxy, propoxy, isopropoxy, linear, secondary or tertiary butoxy, isobutoxy groups, and the like;
an aryl group: a monocyclic or bicyclic aromatic group containing between 6 and 10 carbon atoms. By way of examples of an aryl group, mention may be made of phenyl or naphthyl group;
a heteroaryl group: a monocyclic or bicyclic aromatic group containing between 4 and 9 carbon atoms and containing 1 or 2 heteroatoms, selected from an oxygen atom, a nitrogen atom and a sulfur atom, more particularly selected from an oxygen atom and a nitrogen atom, still more particularly nitrogen. By way of examples, mention may be made of, but not limited to pyridinyl group and the like;
a heterocyclyl or heterocyclic group: a ring structure having between 3 to 12 atoms, in particular between 4 and 8 atoms, wherein one or more atoms are selected from the group consisting of a nitrogen atom, an oxygen atom, and a sulfur atom, the remainder of the ring atoms being carbon. More particularly, a heterocyclyl or heterocyclic group may be a ring structure having, unless otherwise mentioned, between 4 and 9 carbon atoms, in particular 4 and 8, and containing 1 or 2 heteroatoms selected from an oxygen atom, a nitrogen atom and a sulfur atom, in particular an oxygen atom and a nitrogen atom. The heterocyclic group may be a monocyclic, a bicyclic, a spirocyclic or a bridged ring system. The heterocyclic group is optionally substituted on carbon or nitrogen at one or more positions. By way of examples, mention may be made of, but not limited to pyrrolidine, piperazine, piperidine, tetrahydropyran, morpholine, diazepane group, and the like;
an alkylene group: a linear or branched, saturated divalent alkyl group. More particularly, a (Cx-Cy) alkylene group, where x and y are integers, x<y, is a linear or branched, saturated divalent alkyl group comprising from x to y carbon atoms. For example, a (C1-C3) alkylene group represents a linear or branched divalent carbon-based chain of 1 to 3 carbon atoms. By way of examples, mention may be made of, but not limited to, a methylene group, an ethylene group, a 1-methylethylene group, a propylene group, and the like; 20
an alkylthio group: a radical —S-alkyl in which the alkyl group is as defined above.

More particularly, a (Cx-Cy) alkylthio group, where x and y are integers, x<y, is a radical —S-(Cx-Cy) alkyl group in which the (Cx-Cy) alkyl is as defined above; for example, a (C1-C4) alkylthio group. By way of examples, mention may be made of, but not limited to a methylthio group, an ethylthio group, a propylthio group, a butylthio group, and the like;
a benzyl group: a radical-CH₂-phenyl group;
a dialkylamino group: an amino group substituted with two alkyl groups, in which the alkyl group is as defined above;
a sulfonyl group: a SO2 group;
an alkylsulfonyl group: a radical —SO2-alkyl in which the alkyl group is as defined above. More particularly, a (Cx-Cy) alkylsulfonyl group, where x and y are integers, x<y, is a radical —SO2-(Cx-Cy) alkyl group in which the (Cx-Cy) alkyl is as defined above; for example, a (C1-C4) alkylsulfonyl group. By way of examples, mention may be made of, but not limited to a methylsulfonyl group, an ethylsulfonyl group, a propylsulfonyl group, a butylsulfonyl group, and the like;
a silyl group: a group containing a silicon atom. Example of silyl groups includes trimethylsilyl group.

All patents, patent applications, and publications referred herein are incorporated by reference.

### KRAS G12C Inhibitors

KRAS G12C inhibitors are compounds that inhibit the KRAS G12C mutant protein. In particular, the KRAS G12C inhibitors used in the combination are compounds that negatively modulate or inhibit all or a portion of the enzymatic activity of KRAS G12C.

The KRAS G12C inhibitor may be any KRAS G12C inhibitor known in the art. In particular, it is one of the KRAS G12C inhibitors described in more detail in the following paragraphs.

The therapeutic combination described hereafter comprises one or more KRAS G12C inhibitors.

The KRAS G12C inhibitor, in one specific embodiment, may be selected from the compounds disclosed as KRAS G12C inhibitors in patent applications WO2018/217651, WO2019/213516, WO2018/119183; and patent applications WO2019/99524, WO2017/201161 and WO2020/101736.

In an exemplary embodiment, the KRAS G12C inhibitor may be selected from the group comprising, in particular consisting of, compounds of formula (I), in particular of formula (I′); compounds of formula (II), in particular of formula (II′), as detailed hereinafter; their pharmaceutically acceptable salts; and mixtures thereof.

In an exemplary embodiment, the KRAS G12C inhibitor used in the therapeutic combination is a compound of formula (I) below:

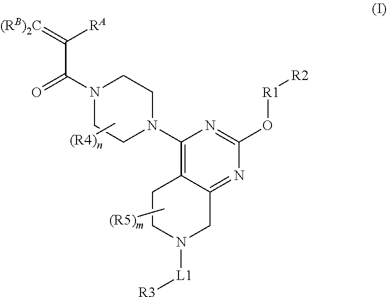

wherein
R^{A} is hydrogen, a (C1-C4) alkyl group or a halogen atom, in particular R^{A} is a fluorine atom;
R^{B} are independently of each other a hydrogen atom or a (C1-C4) alkyl group, in particular both R^{B} are hydrogen atoms;
R1 is a (C1-C3) alkylene group, in particular a methylene group;
R2 is a (C4-C8) heterocyclyl group comprising 1 or 2 heteroatoms selected from an oxygen atom, a nitrogen atom and a sulfur atom, in particular an oxygen atom and a nitrogen atom, and more particularly 1 or 2 nitrogen atom(s), such as a pyrrolidinyl group, unsubstituted or substituted with one or more R6;
L1 is a bond or a (C1-C3) alkylene group, in particular L1 is a bond;
R3 is a (C6-C10) aryl group or a heteroaryl group containing between 4 and 9 carbon atoms and 1 or 2 heteroatoms selected from an oxygen atom, a nitrogen atom and a sulfur atom, unsubstituted or substituted with one or more R7 groups; in particular R3 is a naphthyl group substituted with one or more R7 groups;
n is zero, 1 or 2, and R4 is chosen from (C1-C4) alkyl groups unsubstituted or substituted with one or more cyano group(s) or halogen atom(s);

m is zero, 1 or 2; and R5 is chosen from (C1-C4) alkyl groups unsubstituted or substituted with one or more halogen atoms;

R6 is a (C1-C4) alkyl group and more particularly a methyl group;

R7 is chosen from a halogen atom, in particular a chlorine atom, and a (C1-C4) alkyl group unsubstituted or substituted with one or more halogen atoms;

or a pharmaceutically acceptable salt thereof.

In an exemplary embodiment, the KRAS G12C inhibitor is of formula (I′):

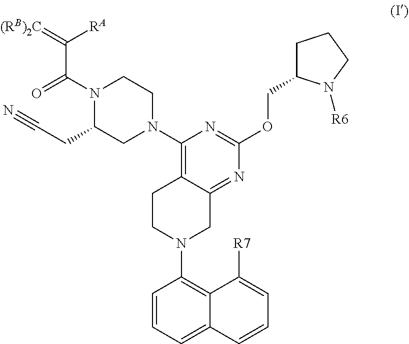

wherein:
R^{A} is a halogen atom, in particular a fluorine atom;
R^{B} are independently of each other hydrogen or a (C1-C4) alkyl; in particular both R^{B} are hydrogen atoms;
R6 is as defined above and more particularly a methyl group; and
R7 is a halogen atom, in particular a chlorine atom;
or a pharmaceutically acceptable salt thereof.

In an exemplary embodiment, the KRAS G12C inhibitor used in the therapeutic combination is 2-((S)-4-(7-(8-chloronaphthalen-1-yl)-2-(((S)-1-methylpyrrolidin-2-yl) methoxy)-5,6,7,8-tetrahydropyrido[3,4-d]pyrimidin-4-yl)-1-(2-fluoroacryloyl) piperazin-2-yl) acetonitrile, in other words the compound of formula:

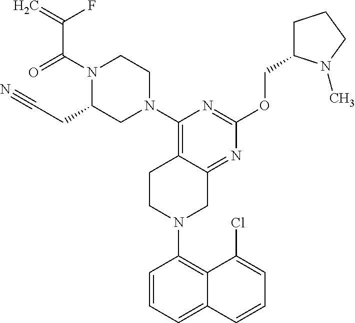 [SMILES: C=C(F)C(=O)N1CCN(c2nc(OC[C@@H]3CCCN3C)nc3c2CCN(c2cccc4cccc(Cl)c24)C3)C[C@@H]1CC#N]

which corresponds to Adagrasib proposed by Mirati Therapeutics, Inc., also known as MRTX849.

In another exemplary embodiment, the KRAS G12C inhibitor used in the therapeutic combination is of formula (II) below:

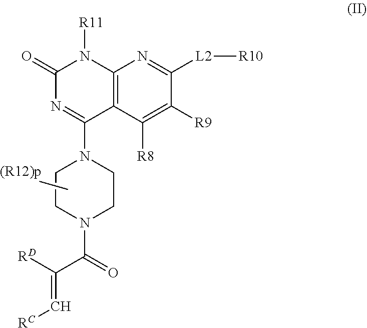

wherein:
R8 is a hydrogen atom, a halogen atom, for example a fluorine atom, or a (C1-C3) alkyl group unsubstituted or substituted with one or more halogen atoms, in particular with one or more fluorine atoms; in particular R8 is a hydrogen atom;
R9 is a halogen atom, in particular a fluorine atom or a (C1-C3) alkyl group unsubstituted or substituted with one or more halogen atoms, in particular with one or more fluorine atoms; in particular R9 is a fluorine atom;
L2 is a single bond or a methylene group; in particular a single bond;
R10 is a (C6-C10) aryl group or a heteroaryl group containing between 4 and 9 carbon atoms and 1 or 2 heteroatoms selected from an oxygen atom, a nitrogen atom and a sulfur atom, in particular a phenyl group, unsubstituted or substituted with one or more R13 groups;
R11 is a (C6-C10) aryl group or a heteroaryl group containing between 4 and 9 carbon atoms and 1 or 2 heteroatoms selected from an oxygen atom, a nitrogen atom and a sulfur atom, unsubstituted or substituted, preferably in ortho positions, with one or more R14 groups;
R^{C} is a hydrogen atom or a (C1-C3) alkyl group;
R^{D} is a hydrogen atom, a (C1-C3) alkyl group or a halogen atom in particular a fluorine atom;
p is zero, 1 or 2; and R12 is a (C1-C3) alkyl group, in particular a methyl group;
R13 is chosen from a halogen atom, in particular a fluorine atom, a hydroxyl group (OH) and a (C1-C3) alkoxy group;
R14 is chosen from a hydrogen atom, a (C1-C4) alkyl group and a (C3-C6) cycloalkyl group;
or a pharmaceutically acceptable salt thereof.

Among the KRAS G12C inhibitors of formula (II), mention may be made in particular of the compounds for which R10 is the following group:

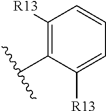

in particular a group

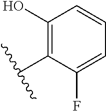 [SMILES: *c1c(O)cccc1F]

Among the KRAS G12C inhibitors of formula (II), mention may be made in particular of the compounds for which R11 is a group:

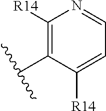

in particular a group

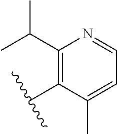 [SMILES: *c1c(C)ccnc1C(C)C]

In an exemplary embodiment, the KRAS G12C inhibitor is of formula (II′) below:

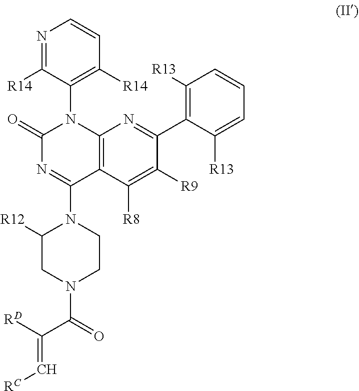

wherein:
R^{C} and R^{D} are as defined above, in particular both R^{C} and R^{D} are hydrogen atoms,
R8 is as defined above, in particular R8 is a hydrogen atom,
R9 is as defined above, in particular a halogen atom and more particularly a fluorine atom,
R12 is as defined above, in particular a methyl group,
R13 are as defined above, in particular one is a hydroxyl group and the other a fluorine atom,
R14 are as defined above, in particular (C1-C3) alkyl groups; and more particularly one is a methyl group and the other an isopropyl group,
or a pharmaceutically acceptable salt thereof.

In an exemplary embodiment, the KRAS G12C inhibitor used in the therapeutic combination is 4-((S)-4-acryloyl-2-methylpiperazin-1-yl)-6-fluoro-7-(2-fluoro-6-hydroxyphenyl)-1-(2-isopropyl-4-methylpyridin-3-yl) pyrido[2,3-d]pyrimidin-2 (1H)-one, in other words the compound of formula:

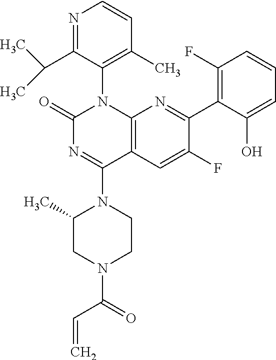 [SMILES: C=CC(=O)N1CCN(c2nc(=O)n(-c3c(C)ccnc3C(C)C)c3nc(-c4c(O)cccc4F)c(F)cc23)[C@@H](C)C1]

which corresponds to Sotorasib proposed by Amgen, also known as AMG 510.

In an exemplary embodiment, the KRAS G12C inhibitor used in the therapeutic combination is:
2-((S)-4-(7-(8-chloronaphthalen-1-yl)-2-(((S)-1-methylpyrrolidin-2-yl) methoxy)-5,6,7,8-tetrahydropyrido[3,4-d]pyrimidin-4-yl)-1-(2-fluoroacryloyl) piperazin-2-yl) acetonitrile (Adagrasib, also known as MRTX849);
4-((S)-4-acryloyl-2-methylpiperazin-1-yl)-6-fluoro-7-(2-fluoro-6-hydroxyphenyl)-1-(2-isopropyl-4-methylpyridin-3-yl) pyrido[2,3-d]pyrimidin-2 (1H)-one (Sotorasib, also known as AMG-510);
or a pharmaceutically acceptable salt thereof.

In an exemplary embodiment, Adagrasib (MRTX849) is the KRAS G12C used in the therapeutic combination.

In another exemplary embodiment, Sotorasib (AMG-510) is the KRAS G12C used in the therapeutic combination.

### TEAD Inhibitors

TEAD inhibitors are compounds that have an inhibitory activity of YAP1/TAZ-TEAD or TEAD-dependent gene transcription.

The therapeutic combination described hereinafter comprises one or more specific TEAD inhibitors as disclosed hereafter.

The TEAD inhibitor used in the therapeutic combination of the present disclosure is selected from the group comprising, in particular consisting of, molecules of formula (III), indole compounds of formula (IV), indane compounds of formula (V), as detailed hereinafter, their pharmaceutically acceptable salts, and mixtures thereof.

In an exemplary embodiment, the TEAD inhibitor used in the therapeutic combination is of formula (III) below

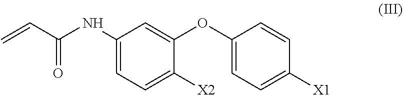

wherein
X1 is a halogen atom, in particular a chlorine atom;
X2 is a (C1-C4) alkyl group, in particular a methyl group;
or a pharmaceutically acceptable salt thereof.

For example, mention may be made of the N-(3-(4-chlorophenoxy)-4-methylphenyl) acrylamide, also known as K-975.

In another exemplary embodiment, the TEAD inhibitor used in the therapeutic combination is selected from the indole compounds disclosed in the patent application WO2021/204823. Thus, in an exemplary embodiment, the TEAD inhibitor is of formula (IV) below

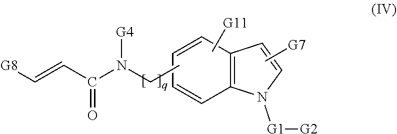

wherein
q is an integer chosen from 0 and 1;
G1 is chosen from:
   a single bond, and
   a (C1-C4) alkylene group, in particular a methylene group,
G2 is chosen from:
   a (C1-C4) alkyl group unsubstituted or substituted with one or more fluorine atoms,
   a (C1-C3) alkoxy group substituted with one or more fluorine atoms,
   a phenyl group unsubstituted or substituted with one or more G3 groups,
   a (C4-C8) cycloalkyl group unsubstituted or substituted with one or more G5 groups,
   a (C4-C8) heterocyclyl group containing 1 or 2 heteroatoms selected from an oxygen atom and a nitrogen atom, unsubstituted or substituted with one or more G6 groups, and
   a NG9G10 group,
G3 is chosen from:
   a (C1-C4) alkyl group, in particular a methyl group, unsubstituted or substituted with one or more fluorine atoms,
   a cyclopropyl group,
   a halogen atom,
   a (C1-C3) alkoxy group unsubstituted or substituted with one or more fluorine atoms,
   a pentafluorosulfanyl group,
   a nitrile group,
   a (C1-C3) trialkylsilyl group,
   a (C1-C3) alkylsulfonyl group, and
   a phenyl group unsubstituted or substituted with a trifluoromethyl group,
G4 is chosen from a hydrogen atom and a (C1-C4) alkyl group,
G5 is chosen from a fluorine atom and a trifluoromethyl group,
G6 is chosen from:
   a phenyl group unsubstituted or substituted with one or more fluorine atoms or one or more CF3 groups,
   a (C1-C4) alkyl group substituted with one or more fluorine atoms, and
   a fluorine atom,
G7 is chosen from:
   a hydrogen atom,
   a nitrile group,
   a (C1-C4) alkyl group, in particular a methyl group, unsubstituted or substituted with
   a (C1-C3) alkoxy group, in particular a methoxy group, or a hydroxy group,
   a COO(C1-C4) alkyl group, and
   a CONH2 group,
G8 is chosen from a hydrogen atom and a (C1-C4) alkyl group unsubstituted or substituted with a di(C1-C4) alkylamino group,
G9 and G10 are identical or different and chosen from (C1-C3) alkyl group unsubstituted
or substituted with one or more fluorine atoms,
G11 is chosen from a hydrogen atom, a fluorine atom and a chlorine atom;
or a pharmaceutically acceptable salt thereof.

In one aspect, the TEAD inhibitor is of formula (IV′) below

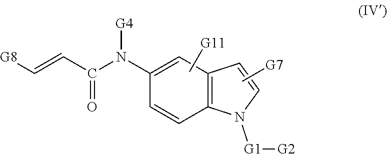

wherein G1, G2, G4, G7, G8 and G11 are as defined above.

Among the TEAD inhibitors of formula (IV) or (IV′), mention may be made of the compounds for which G4 is a hydrogen atom.

Among the TEAD inhibitors of formula (IV) or (IV′), mention may be made of the compounds for which G7 is a hydrogen atom or a (C1-C4) alkyl group, especially a methyl group, unsubstituted or substituted with a (C1-C3) alkoxy group, especially a methoxy group.

Among the TEAD inhibitors of formula (IV) or (IV′), mention may be made of the compounds for which G8 is a hydrogen atom.

Among the TEAD inhibitors of formula (IV) or (IV′), mention may be made of the compounds for which G11 is a hydrogen atom.

Among the TEAD inhibitors of formula (IV), mention may be made of the compounds for which:
q is 0,
G1 is a single bond or a methylene group,
G2 is chosen from:
   a phenyl group unsubstituted or substituted with one or more G3 groups,
   a (C4-C8) cycloalkyl group unsubstituted or substituted with one or more G5 groups, and
   a (C4-C8) heterocyclyl group containing 1 or 2 heteroatoms selected from an oxygen atom and a nitrogen atom, unsubstituted or substituted with one or more G6 groups,
or a pharmaceutically acceptable salt thereof.

In one aspect, G2 is a phenyl group substituted with one or more G3 groups, G3 being in particular a (C1-C4) alkyl group substituted with one or more fluorine atoms, in particular a trifluoromethyl group.

In a particular embodiment, the TEAD inhibitor is of formula (IV), in which:
q is 0,
G1 is a single bond or a methylene group,
G2 is chosen from a phenyl group unsubstituted or substituted with one or more G3 groups,
G3 a (C1-C4) alkyl group substituted with one or more fluorine atoms, in particular a trifluoromethyl group;
G4 is a hydrogen atom;
G7 is a hydrogen atom or a (C1-C4) alkyl group, especially a methyl group, unsubstituted or substituted with a (C1-C3) alkoxy group, especially a methoxy group;
G8 is a hydrogen atom; and
G11 is a hydrogen atom;
or a pharmaceutically acceptable salt thereof.

For example, among the TEAD inhibitors of formula (IV), mention may be made in particular of the following compounds:
N-(1-(4-(trifluoromethyl)phenyl)-1H-indol-5-yl) acrylamide,
N-(3-(methoxymethyl)-1-(4-(trifluoromethyl)phenyl)-1H-indol-5-yl) acrylamide,
N-(3-methyl-1-(3-(trifluoromethyl)benzyl)-1H-indol-5-yl) acrylamide, and their pharmaceutical acceptable salts thereof.

The TEAD inhibitors of formula (IV) may be prepared according to the processes as disclosed in the above-referenced patent application WO2021/204823.

In another exemplary embodiment, the TEAD inhibitor used in the therapeutic combination is selected from the indane compounds disclosed the patent application WO2022/023460 filed by SANOFI.

Thus, in an exemplary embodiment, the TEAD inhibitor is of formula (V)

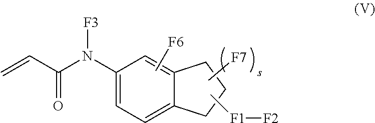

wherein:
F1 is chosen from:
   an oxygen atom, and
   a group —N(H)— or a group —N(F8)-;
wherein F8 is a (C1-C4) alkyl group, in particular a methyl group;
F2 is chosen from:
   a phenyl group unsubstituted or substituted with one or more F4 groups;
   a benzyl group unsubstituted or substituted with one or more F5 groups;
   a (C4-C8) cycloalkyl group, in particular a cyclohexyl group or a cyclopentyl group and more particularly a cyclohexyl group, said (C4-C8) cycloalkyl group being unsubstituted or substituted with one or more F5 groups;
   a heteroaryl group, containing between 4 and 9 carbon atoms and 1 or 2 heteroatoms selected from an oxygen atom and a nitrogen atom, in particular a pyridinyl group, said heteroaryl group being unsubstituted or substituted with one or more F5 groups;
   a (C1-C6) alkyl group, in particular a (C1-C4) alkyl group, said (C1-C6) alkyl group being substituted with one or more fluorine atoms;
F3 is chosen from:
   a hydrogen atom, and
   a (C1-C4) alkyl group, in particular a methyl group;
F4 is chosen from:
   a halogen atom, in particular a fluorine atom or a chlorine atom;
   a (C1-C4) alkyl group unsubstituted or substituted with one or more fluorine atoms, in particular a methyl group or a trifluoromethyl group;
   a (C1-C4) alkoxy group unsubstituted or substituted with one or more fluorine atoms, in particular a methoxy group or a trifluoromethoxy group;
   a C(O)—O—(C1-C4) alkyl group, in particular a C(O)—O-methyl group;
   a (C3-C6) cycloalkyl group; in particular cyclopropyl group;
   a (C1-C4) alkylthio group; in particular a methylthio group; and
   a pentafluorosulfanyl group;
F5 is chosen from:
   a halogen atom, in particular a fluorine atom; and
   a (C1-C4) alkyl group unsubstituted or substituted with one or more fluorine atoms, in particular a trifluoromethyl group;
F6 is chosen from a hydrogen atom and a halogen atom, in particular a fluorine atom;
F7 is independently chosen from:
   a halogen atom, in particular a fluorine atom;
   a (C1-C4) alkyl group, in particular a methyl group;
   a hydroxy group;
   a (C1-C4) alkoxy group, in particular a methoxy group;
   s is 0, 1 or 2;
or a pharmaceutically acceptable salt thereof

In a particular embodiment, the TEAD inhibitor is of formula (V′)

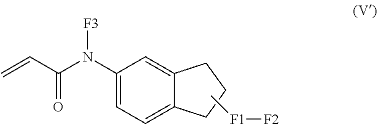

wherein:
F1 is chosen from:
   an oxygen atom, and
   a group —N(H)—;
F2 is chosen from:
   a phenyl group unsubstituted or substituted with one or more F4 groups;
   a benzyl group unsubstituted or substituted with one or more F5 groups;
   a (C4-C8) cycloalkyl group, in particular a cyclohexyl group, said (C4-C8) cycloalkyl group being unsubstituted or substituted with one or more F5 groups;
   a heteroaryl group containing between 4 and 9 carbon atoms and 1 or 2 heteroatoms selected from an oxygen atom and a nitrogen atom, in particular a pyridinyl group, said heteroaryl group being unsubstituted or substituted with one or more F5 groups;
   a (C1-C4) alkyl group substituted with one or more fluorine atoms, in particular a (C2-C4) alkyl group substituted with one or more fluorine atoms, more particularly a (C2-C3) alkyl group substituted with a trifluoromethyl group;
F3 is chosen from:
   a hydrogen atom, and
   a (C1-C4) alkyl group, in particular a methyl group;
F4 is chosen from:
   a halogen atom, in particular a fluorine atom;
   a (C1-C4) alkyl group unsubstituted or substituted with one or more fluorine atoms, in particular a methyl group or a trifluoromethyl group;
   a (C1-C4) alkoxy group unsubstituted or substituted with one or more fluorine atoms, in particular a methoxy group or a trifluoromethoxy group; and
   a —C(O)—O—(C1-C3) alkyl group, in particular a —C(O)—O-methyl group;
F5 is chosen from:
   a halogen atom, in particular a fluorine atom; and
   a (C1-C4) alkyl group unsubstituted or substituted with one or more fluorine atoms, in particular a trifluoromethyl group;
or a pharmaceutically acceptable salt thereof.

In one aspect, the TEAD inhibitor is of formula (V) in which the indane is substituted with the radical -F1-F2 in position 1 according to the IUPAC numbering, in other words is a compound of the following formula (Va):

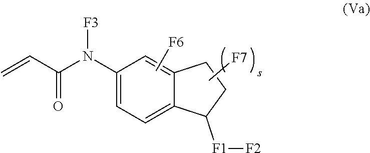

or a pharmaceutically acceptable salt thereof, wherein F1, F2, F3, F6, F7 and s are as defined in the present disclosure.

In particular, the TEAD inhibitor is of formula (V′) in which the indane is substituted with the radical -F1-F2 in position 1 according to the IUPAC numbering, in other words is a compound of the following formula (V′a):

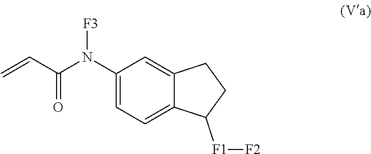

or a pharmaceutically acceptable salt thereof, wherein F1, F2, and F3 are as defined in the present disclosure.

In another aspect, the TEAD inhibitor is of formula (V) in which the indane is substituted with the radical -F1-F2 in position 3 according to the IUPAC numbering, in other words is a compound of the following formula (Vb):

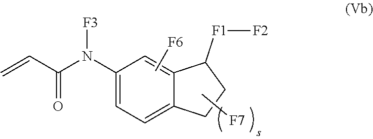

or a pharmaceutically acceptable salt thereof, wherein F1, F2, F3, F6, F7 and s are as defined in the present disclosure.

In particular, the TEAD inhibitor is of formula (V′) in which the indane is substituted with the radical -F1-F2 in position 3 according to the IUPAC numbering, in other words is a compound of the following formula (V′b):

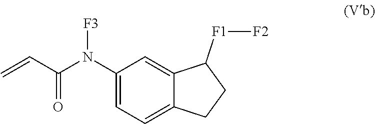

or a pharmaceutically acceptable salt thereof, wherein F1, F2, and F3 are as defined in the present disclosure.

In a particular embodiment, the TEAD inhibitor is of formula (V), in particular of formula (V′), or of any sub-formula (Va), (Vb), (V′a) or (V′b) thereof, in which F3 is a hydrogen atom.

In a particular embodiment, the TEAD is of formula (V), in particular of formula (V′), or of any sub-formula (Va), (Vb), (V′a) or (V′b) thereof, in which:
F1 is an oxygen atom and F2 is chosen from:
   a phenyl group unsubstituted or substituted with one or more F4 groups;
   a (C4-C8) cycloalkyl group, in particular a cyclohexyl group or a cyclopentyl group, said cycloalkyl group being unsubstituted or substituted with one or more F5 groups; and
   a (C1-C5) alkyl group, in particular a (C1-C4) alkyl group, said alkyl group being substituted with one or more fluorine atoms, especially a (C1-C4) alkyl group, in particular a (C2-C3) alkyl group, substituted with a trifluoromethyl group;
or F1 is —N(H)— and F2 is chosen from:
   a phenyl group unsubstituted or substituted with one or more F4 groups; and
   a heteroaryl group containing between 4 and 9 carbon atoms and 1 or 2 heteroatoms selected from oxygen and nitrogen, in particular a pyridinyl group, said heteroaryl group being unsubstituted or substituted with one or more F5 groups.

In a particular embodiment, the TEAD inhibitor is of formula (V), in particular of formula (V′), or of any sub-formula (Va), (Vb), (V′a) or (V′b) thereof, in which F2 is chosen from:
a phenyl group unsubstituted or substituted with one or more F4 groups; and
a (C4-C8) cycloalkyl group, in particular a cyclohexyl group, said cycloalkyl group being unsubstituted or substituted with one or more F5 groups, provided that F1 is an oxygen atom.

Among the TEAD inhibitors of formula (V), in particular of formula (V′), or of any sub-formula (Va), (Vb), (V′a) or (V′b) thereof, mention may be made in particular of the compounds for which:
F1 is an oxygen atom, and
F2 is a (C4-C8) cycloalkyl group, in particular a cyclohexyl group, substituted with on or more F5 groups, in particular said F5 groups being chosen from a (C1-C4) alkyl group substituted with one or more fluorine atoms, in particular a trifluoromethyl group.

Among the TEAD inhibitors of formula (V), in particular of formula (V′), or of any sub-formula (Va), (Vb), (V′a) or (V′b) thereof, mention may be made in particular of the compounds for which
F1 is a —N(H)— group, and
F2 is a phenyl group substituted with one or more F4 groups, in particular said F4 groups being chosen from a (C1-C4) alkyl group substituted with one or more fluorine atoms, in particular a trifluoromethyl group, or a halogen atom, in particular a fluorine atom.

In particular, in the compounds of formula (V), in particular of formula (V′), or of any sub-formula (Va), (Vb), (V′a) or (V′b) thereof, the F4 group(s) is (are) in the meta and/or para position(s) of the F2 phenyl group.

In particular, in the compounds of formula (V), in particular of formula (V′), or of any sub-formula (Va), (Vb), (V′a) or (V′b) thereof, the F5 group(s) is (are) in the para and/or meta position(s), in particular in the para position of the F2 group.

In a specific embodiment, the TEAD inhibitor is of formula (V′), in particular of formula (V′a) and (V′b), for which:
F1 is chosen from an oxygen atom and a group —N(H)—;
F2 is chosen from:
   a phenyl group unsubstituted or substituted with one or more F4 groups;
   a benzyl group unsubstituted or substituted with one or more trifluoromethyl groups; provided that F1 is —N(H)—;
   a cyclohexyl group substituted with one or more F5 groups;
   a pyridinyl group substituted with one or more F5 groups; and
   a (C2-C4) alkyl group substituted with one or more fluorine atoms, in particular a (C2-C3) alkyl group substituted with a trifluoromethyl group;
F3 is chosen from a hydrogen atom and a methyl group;
F4 is chosen from a fluorine atom, a methyl group, a trifluoromethyl group, a methoxy group, a trifluoromethoxy group and a —C(O)—O-methyl group; and
F5 is chosen from a fluorine atom and a trifluoromethyl group;
or a pharmaceutically acceptable salt thereof.

In a particular embodiment, the TEAD inhibitor is of formula (V), in particular of formula (Va) or (Vb), for which:
F1 is an oxygen atom or a group —N(H)—;
F2 is chosen from:
   a phenyl group unsubstituted or substituted with one or more F4 groups;
   a (C4-C8) cycloalkyl group, in particular a cyclohexyl group, said (C4-C8) cycloalkyl group being unsubstituted or substituted with one or more F5 groups;
F3 is a hydrogen atom or a methyl group; in particular F3 is a hydrogen atom;
F4 is a (C1-C4) alkyl group substituted with one or more fluorine atoms, in particular a trifluoromethyl group, or a halogen atom, in particular a fluorine atom;
F5 is a (C1-C4) alkyl group substituted with one or more fluorine atoms, in particular a trifluoromethyl group;
F6 is a hydrogen atom; and
s is 0;
or a pharmaceutically acceptable salt thereof.

For example, among the TEAD inhibitors of formula (V), mention may be made in particular of the following compounds:
N-(1-((3-(trifluoromethyl)phenyl)amino)-2,3-dihydro-1H-inden-5-yl) acrylamide;
N-(3-(((trans)-4-(trifluoromethyl)cyclohexyl)oxy)-2,3-dihydro-1H-inden-5-yl) acrylamide;
N-(3-((3,4-difluorophenyl)amino)-2,3-dihydro-1H-inden-5-yl) acrylamide;
N-(3-(4-(trifluoromethyl) phenoxy)-2,3-dihydro-1H-inden-5-yl) acrylamide;
and their pharmaceutically acceptable salts thereof.

The TEAD inhibitors of formula (V) may be prepared according to the processes as disclosed in the above-referenced patent application WO2022/023460.

In an exemplary embodiment, the therapeutic combination uses as the TEAD inhibitor:
N-(3-(4-chlorophenoxy)-4-methylphenyl) acrylamide, also known as K-975;
N-(1-(4-(trifluoromethyl)phenyl)-1H-indol-5-yl) acrylamide;
N-(3-(methoxymethyl)-1-(4-(trifluoromethyl)phenyl)-1H-indol-5-yl) acrylamide;
N-(3-methyl-1-(3-(trifluoromethyl)benzyl)-1H-indol-5-yl) acrylamide;
N-(1-((3-(trifluoromethyl)phenyl)amino)-2,3-dihydro-1H-inden-5-yl) acrylamide;
N-(3-(((trans)-4-(trifluoromethyl)cyclohexyl)oxy)-2,3-dihydro-1H-inden-5-yl) acrylamide;
N-(3-((3,4-difluorophenyl)amino)-2,3-dihydro-1H-inden-5-yl) acrylamide;
N-(3-(4-(trifluoromethyl) phenoxy)-2,3-dihydro-1H-inden-5-yl) acrylamide;
or a pharmaceutically acceptable salt thereof.

The compounds of formula (I), (II), (III), (IV) and (V), or of any sub-formula thereof, as described above may comprise one or more asymmetric carbon atoms. They may thus exist in the form of enantiomers or diastereoisomers and also mixtures thereof.

The compounds of formula (I), (II), (III), (IV) and (V), or of any sub-formula thereof, may be present as well under tautomer forms.

The compounds of formula (I), (II), (III), (IV) and (V), or of any sub-formula thereof, may exist in the form of bases or addition salts with acids or bases, in particular pharmaceutically acceptable salts.

All the combinations of one or more of the specific KRAS G12C inhibitors as detailed above and one or more of the specific TEAD inhibitors as detailed above are part of the description.

Thus, the combination may comprise one or more KRAS G12C inhibitors selected from the group comprising, in particular consisting of, compounds of formula (I), compounds of formula (II), compounds of any sub-formula thereof, and pharmaceutically acceptable salts thereof as defined above; and one or more specific TEAD inhibitors selected from the group comprising, in particular consisting of, compounds of formula (III), compounds of formula (IV), compounds of formula (V), compounds of any sub-formula thereof, and pharmaceutically acceptable salts thereof as defined above.

In an exemplary embodiment, the combination may comprise:
one or more of KRAS G12C inhibitors chosen from:
2-((S)-4-(7-(8-chloronaphthalen-1-yl)-2-(((S)-1-methylpyrrolidin-2-yl) methoxy)-5,6,7,8-tetrahydropyrido[3,4-d]pyrimidin-4-yl)-1-(2-fluoroacryloyl) piperazin-2-yl) acetonitrile (Adagrasib, also known as MRTX849);
4-((S)-4-acryloyl-2-methylpiperazin-1-yl)-6-fluoro-7-(2-fluoro-6-hydroxyphenyl)-1-(2-isopropyl-4-methylpyridin-3-yl) pyrido[2,3-d]pyrimidin-2 (1H)-one (Sotorasib, also known as AMG-510);
   or a pharmaceutically acceptable salt thereof;
      and
   one or more of TEAD inhibitors chosen from:
N-(3-(4-chlorophenoxy)-4-methylphenyl) acrylamide, also known as K-975;
N-(1-(4-(trifluoromethyl)phenyl)-1H-indol-5-yl) acrylamide;
N-(3-(methoxymethyl)-1-(4-(trifluoromethyl)phenyl)-1H-indol-5-yl) acrylamide;
N-(3-methyl-1-(3-(trifluoromethyl)benzyl)-1H-indol-5-yl) acrylamide;
N-(1-((3-(trifluoromethyl)phenyl)amino)-2,3-dihydro-1H-inden-5-yl) acrylamide;
N-(3-(((trans)-4-(trifluoromethyl)cyclohexyl)oxy)-2,3-dihydro-1H-inden-5-yl) acrylamide;
N-(3-((3,4-difluorophenyl)amino)-2,3-dihydro-1H-inden-5-yl) acrylamide;
N-(3-(4-(trifluoromethyl) phenoxy)-2,3-dihydro-1H-inden-5-yl) acrylamide;
   or a pharmaceutically acceptable salt thereof.

### Applications

As mentioned above, the inventors have shown that the presence of a specific TEAD inhibitor as described above, in combination with a KRAS G12C inhibitor, makes it possible to drastically enhance the efficiency of the KRAS G12C inhibitor.

Thus, the combination of a TEAD inhibitor as disclosed hereabove and a KRAS G12C inhibitor results in synergistically effect for inhibiting KRAS G12C protein.

The potentiation effect of the addition of said TEAD inhibitor in combination with a KRAS G12C inhibitor advantageously allows to consider the implementation of a reduced dose of the KRAS G12C inhibitor with the same, or even an improved efficacy.

Said KRAS G12C inhibitor and said TEAD inhibitor are thus used in combination therapy. The therapeutic combination of said KRAS G12C inhibitor(s) and said TEAD inhibitor(s) is more particularly for use in treating pathologies involving KRAS G12C, in particular in treating KRAS G12C-associated diseases or disorders, especially KRAS G12C-mediated cancers.

The KRAS G12C-mediated cancers include lung adenocarcinoma, pancreatic ductal adenocarcinoma, rectum adenocarcinoma, colon adenocarcinoma, bile duct carcinoma, chronic myelomonocytic leukemia (CMML), rhabdomyosarcoma, endometrial cancer, bladder cancer, and ovarian cancer.

The combination of said KRAS G12C inhibitor and said TEAD inhibitor may advantageously be used in the treatment of non-small cell lung, small cell lung, pancreatic or colorectal cancer.

Thus, according to another aspect, hereinafter is described a therapeutic combination of KRAS G12C inhibitor(s) and specific TEAD inhibitor(s) as disclosed above for use in the treatment of KRAS G12C-mediated cancers, in particular lung adenocarcinoma, pancreatic ductal adenocarcinoma, rectum adenocarcinoma, colon adenocarcinoma, bile duct carcinoma, chronic myelomonocytic leukemia (CMML), rhabdomyosarcoma, endometrial cancer, bladder cancer, and ovarian cancer; and more particularly of non-small cell lung, small cell lung, pancreatic or colorectal cancer.

The therapeutic combination may be used in the treatment of a patient who has exhibited resistance to prior anti-cancer therapy.

The KRAS G12C inhibitor(s) and TEAD inhibitor(s) used in combination can be administered simultaneously or separately.

The administration of a therapeutic combination can thus include simultaneous administration of the two inhibitors in the same dosage form, simultaneous administration in separate dosage forms, and separate administration.

The KRAS G12C inhibitor is more particularly administered in a therapeutically effective amount. A “therapeutically effective amount or dose” is an amount that is sufficient to negatively modulate or inhibit the activity of KRAS G12C, and thus to ameliorate, or in some manner reduce a symptom or stop or reverse progression of the condition involving KRAS G12C.

A therapeutically effective amount may vary depending upon the subject (e.g. the weight, age and gender of the subject), disease conditions being treated, the severity of the disease condition and the manner of administration.

The TEAD inhibitor is co-administered in combination with the KRAS G12C inhibitor in an amount sufficient to achieve the effect of potentiation of the activity of said KRAS G12C inhibitor. In particular, the TEAD inhibitor in combination therapy is not administered in a therapeutically effective amount. This means that the TEAD inhibitor alone has no effect in the therapeutic treatment, in particular for the treatment of the targeted cancer.

The terms “co-administration” or “administered in combination with” as used herein encompass administration of both KRAS G12C inhibitor(s) and TEAD inhibitor(s) to a subject so that both inhibitors and/or their metabolites are present in the subject at the same time. Co-administration includes combination and pharmaceutical composition not exclusively limited to the ones which are obtained by physical association of the constituents in a single unit dosage, but also to those which allow a separate administration, which can be simultaneous or sequential (also called “spaced out” or “spread out”) over a period of time. Simultaneous administration in separate compositions and administration in a single unit dosage are preferred.

The KRAS G12C inhibitor and TEAD inhibitor of the therapeutic combination may be administered by any method well known in the art. They may be administered by any route, including, without limitation, parenteral, oral, transdermal, and other dosage forms.

The therapeutic combination may also be administered in combination with at least one third active agent, in particular selected from known active agents in anti-cancer therapy.

The therapeutic combination or pharmaceutical composition may also be used in combination with other treatments, such as radiotherapy or chemotherapy.

In one embodiment, both KRAS G12C inhibitor(s) and TEAD inhibitor(s), in particular as described above, can be formulated together in the same dosage form and administered simultaneously.

The combination of KRAS G12C inhibitor(s) and TEAD inhibitor(s) may be used as medicaments, especially medicaments for the treatment of KRAS-G12C-mediated cancers. Thus, there is also described a medicament that comprise at least one KRAS G12C inhibitor and at least one specific TEAD inhibitor as disclosed hereabove.

According to another of its aspects, the present disclosure relates to a pharmaceutical composition comprising the previously described therapeutic combination, in other words comprising at least one KRAS G12C inhibitor in particular as described above and at least one TEAD inhibitor as disclosed hereabove.

In particular, the pharmaceutical composition contains a therapeutically effective dose of said KRAS G12C inhibitor and said TEAD inhibitor in an effective amount to reach a potentiation effect for inhibiting KRAS G12C protein.

In one embodiment, said pharmaceutical composition is for use in the treatment of KRAS G12C-mediated cancers as mentioned above.

The pharmaceutical composition may also contain at least one pharmaceutically acceptable excipient, diluent and/or carrier. Said excipients, diluents and/or carriers are chosen, according to the pharmaceutical form and the desired mode of administration, from the usual excipients, diluents and carriers known to those skilled in the art.

In another embodiment, the KRAS G12C inhibitor(s) and TEAD inhibitor(s) as described above can be simultaneously administered, wherein both the inhibitors are present in separate formulations.

In still another embodiment, the KRAS G12C inhibitor(s) and the TEAD inhibitor(s) can be administered separately, for example the KRAS G12C inhibitor can be administered just followed by the TEAD inhibitor, or vice versa. In some embodiments of the separate administration protocol, the KRAS G12C inhibitor and the TEAD inhibitor are administered a few minutes apart, or a few hours apart, or a few days apart.

The KRAS G12C inhibitor and the TEAD inhibitor when simultaneously administered in separate dosage forms or separately administered can be comprised in distinct pharmaceutical compositions. Said compositions may contain at least one pharmaceutically acceptable excipient, diluent and/or carrier as described above.

According to another of its aspects, the present disclosure relates to a kit comprising in one or more separate packages the previously described therapeutic combination or a pharmaceutical composition comprising both KRAS G12C inhibitor(s) and TEAD inhibitor(s) as disclosed hereabove, optionally together with instructions for administration thereof and/or with a medical device for administration.

The kit is for use in co-administration of said KRAS G12C inhibitor and said TEAD inhibitor, either simultaneously or separately, especially for the treatment of KRAS G12C-mediated cancer, in particular of lung, colorectal or pancreatic cancer.

The KRAS G12C inhibitor(s) and the TEAD inhibitor(s) can be contained in a single pharmaceutical composition or in two separate pharmaceutical compositions in the kit.

According to another aspect, there is also described a method for treating a KRAS G12C-associated cancer, comprising administering the previously described therapeutic combination or pharmaceutical composition to a patient in need thereof.

There is also described a method for treating a KRAS G12C-associated cancer, comprising co-administering to a patient in need thereof a therapeutically effective amount of a KRAS G12C inhibitor and a TEAD inhibitor as disclosed above.

Also provided herein is a use of a therapeutic combination as disclosed herein in the manufacture of a medicament for the treatment of KRAS G12C-mediated cancers.

The examples that follow are not limiting but serve merely to illustrate the present disclosure.

The TEAD inhibitors and KRAS G12C inhibitors used in the examples described hereinafter are the following ones.

### TEAD Inhibitors

TEAD inhibitor N°1: Commercial TEAD K-975;

TEAD inhibitor N°2: N-(1-((3-(trifluoromethyl)phenyl)amino)-2,3-dihydro-1H-inden-5-yl) acrylamide;

TEAD inhibitor N°3: N-(1-(4-(trifluoromethyl)phenyl)-1H-indol-5-yl) acrylamide;

TEAD inhibitor N°4: N-(3-(methoxymethyl)-1-(4-(trifluoromethyl)phenyl)-1H-indol-5-yl) acrylamide;

TEAD inhibitor N°5: N-(3-methyl-1-(3-(trifluoromethyl)benzyl)-1H-indol-5-yl) acrylamide;

TEAD inhibitor N°6: N-(3-(((trans)-4-(trifluoromethyl)cyclohexyl)oxy)-2,3-dihydro-1H-inden-5-yl) acrylamide;

TEAD inhibitor N°7: N-(3-((3,4-difluorophenyl)amino)-2,3-dihydro-1H-inden-5-yl) acrylamide;

TEAD inhibitor N°8: N-(3-(4-(trifluoromethyl) phenoxy)-2,3-dihydro-1H-inden-5-yl) acrylamide.

TEAD inhibitors N°3, N°4 and N°5 were prepared according to the detailed synthesis described in the PCT patent application WO2021/204823.

TEAD inhibitors N°2, N°6, N°7 and N°8 were prepared according to the detailed synthesis described in the PCT patent application WO2022/023460.

### KRAS G12C Inhibitors

KRAS G12C inhibitor N°1: Adagrasib MRTX849 of Mirati Therapeutics, Inc.; KRAS G12C inhibitor N°2: Sotorasib AMG 510 of Amgen.

### EXAMPLE 1: RAY DESIGN COMBINATION EXPERIMENT IN KRAS MUTANT HOP-62 CELL LINE USING THE KRAS G12C INHIBITOR N°1 AND TEAD INHIBITORS N°1 AND N°2

The NSCLC cell line HOP62 which carries a KRASG12C mutation was used for this study. HO-P62 cells were seeded in 384 well plates at 300 cells per well and incubated 24 hours at 37° C. and 5% CO₂. The KRAS G12C inhibitor N°1 used at 14 concentrations ranging from 10,000 nM to 0.006 nM was combined with TEAD inhibitors used at 18 concentrations ranging from 10,000 nM to 0.0001 nM, added to HOP-62 cells and incubated for 96 hours at 37° C. and 5% CO₂. Compound dilution and distribution to cells was performed on the TECAN MCA384 robotic platform. Cell proliferation was measured using Cell Titer Glo from PROMEGA (luminescence).

Three independent experiments with triplicates per experiment were performed and analyzed using the combination analysis software Combo2Screen. TEAD inhibitors were defined as non-active in this cell line, their effect on the KRAS G12C inhibitor was determined as an effect of potentiation and was quantified using the potentiation index based on the following equation:

$$Potention only if Ki = \frac{{IC}_{50} ai}{{IC}_{50} a} < 1$$

IC₅₀ai=IC₅₀ of active compound with inactive compound at one concentration
IC₅₀a=IC₅₀ of active compound alone at one concentration

In this study, the different TEAD inhibitors potentiated the KRAS G12C inhibitor N°1 in HOP-62 KRAS G12C mutant NSCLC cells with a maximum effect of approximately eight-fold, as exemplified below.

TABLE 1

| | | | | Confidence |
|---|---|---|---|---|
| KRAS G12C | TEAD | TEAD | Mean fold | interval fold |
| inhibitor | Inhibitor | inhibitor | potentiation | potentiation |
| N° | N° | concentration | 1/Ki | 1/Ki |
| 1 | 1 | 370 nM | 8 | 29.4; 2.3 |
| 1 | 2 | 370 nM | 8 | 20.4; 3.1 |

### EXAMPLE 2: RAY DESIGN COMBINATION EXPERIMENT IN KRAS MUTANT H-2030 CELL LINES USING THE KRAS G12C INHIBITOR N°1 AND TEAD INHIBITORS N°1 AND N°2

The NSCLC cell line H-2030 which carries a KRASG12C mutation was used for this study. H-2030 cells were seeded in 384 well plates at 250 cells per well and incubated 24 hours at 37° C. and 5% CO₂. The KRAS G12C inhibitor N°1 used at 14 concentrations ranging from 10,000 nM to 0.006 nM was combined with TEAD inhibitors used at 18 concentrations ranging from 10,000 nM to 0.0001 nM, added to H-2030 cells and incubated for 144 hours at 37° C. and 5% CO₂. Compound dilution and distribution to cells was performed on the TECAN MCA384 robotic platform. Cell proliferation was measured using Cell Titer Glo from PROMEGA (luminescence).

Three independent experiments with triplicates/experiment were performed and analyzed using the combination analysis software Combo2Screen. TEAD inhibitors were defined as non-active in this cell line, their effect on the KRAS G12C inhibitor was determined as an effect of potentiation and was quantified using the potentiation index based on the following equation:

$$Potention only if Ki = \frac{{IC}_{50} ai}{{IC}_{50} a} < 1$$

IC₅₀ai=IC₅₀ of active compound with inactive compound at one concentration
IC₅₀a=IC₅₀ of active compound alone at one concentration

In this study, the different TEAD inhibitors potentiated the KRASG12C inhibitor N°1 in H-2030 KRAS G12C mutant NSCLC cells with a maximum effect of approximately 18- to 33-fold, as exemplified below.

TABLE 2

| | | | | Confidence |
|---|---|---|---|---|
| KRAS G12C | TEAD | TEAD | Mean fold | interval fold |
| inhibitor | Inhibitor | inhibitor | potentiation | potentiation |
| N° | N° | concentration | 1/Ki | 1/Ki |
| 1 | 1 | 41 nM | 33 | 396.7; 2.7 |
| 1 | 2 | 41 nM | 18 | 68.5; 5.0 |

### EXAMPLE 3: IC50 SHIFT EXPERIMENT IN THE KRAS G12C MUTANT NSCLC CELL LINE HOP-62 USING THE KRAS G12C INHIBITORS N°1 AND N°2 AND A SET OF STRUCTURALLY DIFFERENT TEAD INHIBITORS

The KRAS G12C mutant NSCLC cell line HOP-62 was used for this study. Cells were seeded in 96 well plates at 1250 cells per well and incubated 24 hours at 37° C. and 5% CO₂. The KRAS G12C inhibitors N°1 and N°2 were used at 10 concentrations and combined with TEAD inhibitors used at 2 concentrations. Compound dilution and distribution to cells was performed on the TECAN MCA384 robotic platform. Cells were incubated with inhibitors for 144 hours at 37° C. and 5% CO₂. Cell proliferation was measured using Cell Titer Glo from PROMEGA (luminescence). For each KRAS G12C inhibitor, IC50 values in these cell lines were determined in the absence or presence of TEAD inhibitors.

In this study, the different TEAD inhibitors potentiate the IC50s of the KRAS G12C inhibitor N°1 or N°2 by up to 21-fold in HOP-62 KRAS G12C mutant NSCLC cells.

TABLE 3
Combination of TEAD inhibitors with KRAS G12C inhibitor No1 (at 0.1 and 1 μM for
the TEAD inhibitors and at ten concentrations from 10,000 nM to 0.3 nM with
3-fold dilution steps for IC50 determination of the KRAS G12C inhibitor)

| | | | +TEADi | +TEADi | | |
|---|---|---|---|---|---|---|
| | | no TEADi | 0.1 μM | 1 μM | KRAS G12Ci | KRAS G12Ci |
| | IC50 | IC50 | IC50 | IC50 | No1 fold | No1 fold |
| TEADi | TEADi | KRAS G12Ci | KRAS G12Ci | KRAS G12Ci | change | change |
| No | (nM) | No1 (nM)⁽*⁾ | No1 (nM) | No1 (nM) | vs 0.1 μM | vs 1 μM |
| 1 | 8800 | 63 | 8 | 7 | 8 | 9 |
| 3 | >10 000 | 63 | 10 | 9 | 6 | 7 |
| 4 | >10 000 | 63 | 12 | 6 | 5 | 10 |
| 5 | >10 000 | 63 | 14 | 9 | 4 | 7 |
| 6 | >10 000 | 63 | 17 | 11 | 4 | 6 |
| 7 | >10 000 | 63 | 11 | 5 | 6 | 13 |
| 8 | >10 000 | 63 | 10 | 7 | 6 | 9 |

⁽*⁾geometric mean value on multiple sets of measures.

TABLE 4
Combination of TEAD inhibitors with KRAS G12C inhibitor No2 (at 0.1 and 1 μM for
the TEAD inhibitors and at ten concentrations from 10,000 nM to 0.3 nM with
3-fold dilution steps for IC50 determination of the KRAS G12C inhibitor)

| | | | +TEADi | +TEADi | | |
|---|---|---|---|---|---|---|
| | | no TEADi | 0.1 μM | 1 μM | KRAS G12Ci | KRAS G12Ci |
| | IC50 | IC50 | IC50 | IC50 | No2 fold | No2 fold |
| TEADi | TEADi | KRAS G12Ci | KRAS G12Ci | KRAS G12Ci | change | change |
| No | (nM) | No2 (nM) | No2 (nM) | No2 (nM) | vs 0.1 μM | vs 1 μM |
| 1 | 8800 | 111 | ND | ND | ND | ND |
| 3 | >10 000 | 111 | 13 | 14 | 9 | 8 |
| 4 | >10 000 | 111 | 14 | 9 | 8 | 12 |
| 5 | >10 000 | 111 | 15 | 9 | 8 | 13 |
| 6 | >10 000 | 111 | 19 | 15 | 6 | 8 |
| 7 | >10 000 | 111 | 12 | 5 | 9 | 21 |
| 8 | >10 000 | 111 | 11 | 9 | 10 | 12 |

These tests on tumor models with KRAS G12C mutations show that the combination of specific TEAD inhibitors of the present disclosure with KRAS G12C inhibitors potentiates the effect of the KRAS G12C inhibitor by several orders of magnitude, in the above examples, between four to 33-fold, depending on the combination of inhibitors and cell lines used.

The potentiation effects are observed in multiple different KRAS G12C models and essay set ups.

1. A therapeutic combination comprising at least one KRAS G12C inhibitor and at least one TEAD inhibitor, wherein the at least one TEAD inhibitor is selected from the group consisting of:
compounds of formula (III) or a pharmaceutically acceptable salt thereof 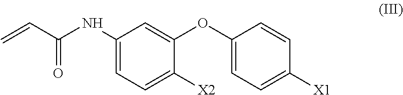
wherein
X1 is a halogen atom, in particular a chlorine atom;
X2 is a (C1-C4) alkyl group, in particular a methyl group;
compounds of formula (IV) or a pharmaceutically acceptable salt thereof 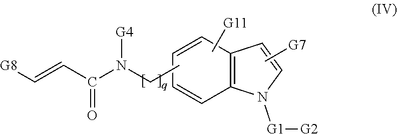
wherein
q is an integer chosen from 0 and 1;
G1 is selected from the group consisting of:
a single bond, and
a (C1-C4) alkylene group, in particular a methylene group,
G2 is selected from the group consisting of:
a (C1-C4) alkyl group unsubstituted or substituted with one or more fluorine atoms,
a (C1-C3) alkoxy group substituted with one or more fluorine atoms,
a phenyl group unsubstituted or substituted with one or more G3 groups,
a (C4-C8) cycloalkyl group unsubstituted or substituted with one or more G5 groups,
a (C4-C8) heterocyclyl group containing 1 or 2 heteroatoms selected from an oxygen atom and a nitrogen atom, unsubstituted or substituted with one or more G6 groups, and
a NG9G10 group,
G3 is selected from the group consisting of:
a (C1-C4) alkyl group, in particular a methyl group, unsubstituted or substituted with one or more fluorine atoms,
a cyclopropyl group,
a halogen atom,
a (C1-C3) alkoxy group unsubstituted or substituted with one or more fluorine atoms,
a pentafluorosulfanyl group,
a nitrile group,
a (C1-C3) trialkylsilyl group,
a (C1-C3) alkylsulfonyl group, and
a phenyl group unsubstituted or substituted with a trifluoromethyl group,
G4 is selected from the group consisting of a hydrogen atom and a (C1-C4) alkyl group,
G5 is selected from the group consisting of a fluorine atom and a trifluoromethyl group,
G6 is selected from the group consisting of:
a phenyl group unsubstituted or substituted with one or more fluorine atoms or one or more CF3 groups,
a (C1-C4) alkyl group substituted with one or more fluorine atoms, and
a fluorine atom,
G7 is selected from the group consisting of:
a hydrogen atom,
a nitrile group,
a (C1-C4) alkyl group, in particular a methyl group, unsubstituted or substituted with a (C1-C3) alkoxy group, in particular a methoxy group, or a hydroxy group,
a COO(C1-C4) alkyl group, and
a CONH2 group,
G8 is selected from the group consisting of a hydrogen atom and a (C1-C4) alkyl group unsubstituted or substituted with a di(C1-C4) alkylamino group,
G9 and G10 are identical or different and chosen from (C1-C3) alkyl group unsubstituted or substituted with one or more fluorine atoms,
G11 is selected from the group consisting of a hydrogen atom, a fluorine atom and a chlorine atom;
compounds of formula (V) or a pharmaceutically acceptable salt thereof 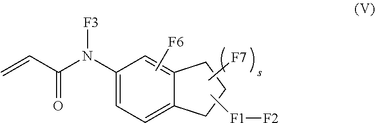
wherein:
F1 is selected from the group consisting of:
an oxygen atom, and
a group —N(H)— or a group —N(F8)-;
wherein F8 is a (C1-C4) alkyl group, in particular a methyl group;
F2 is selected from the group consisting of:
a phenyl group unsubstituted or substituted with one or more F4 groups;
a benzyl group unsubstituted or substituted with one or more F5 groups;
a (C4-C8) cycloalkyl group, in particular a cyclohexyl group or a cyclopentyl group, and more particularly a cyclohexyl group, said (C4-C8) cycloalkyl group being unsubstituted or substituted with one or more F5 groups;
a heteroaryl group, containing between 4 and 9 carbon atoms and 1 or 2 heteroatoms selected from an oxygen atom and a nitrogen atom, in particular a pyridinyl group, said heteroaryl group being unsubstituted or substituted with one or more F5 groups;
a (C1-C6) alkyl group, in particular a (C1-C4) alkyl group, said (C1-C6) alkyl group being substituted with one or more fluorine atoms;
F3 is selected from the group consisting of:
a hydrogen atom, and
a (C1-C4) alkyl group, in particular a methyl group;
F4 is selected from the group consisting of:
a halogen atom, in particular a fluorine atom or a chlorine atom;
a (C1-C4) alkyl group unsubstituted or substituted with one or more fluorine atoms, in particular a methyl group or a trifluoromethyl group;
a (C1-C4) alkoxy group unsubstituted or substituted with one or more fluorine atoms, in particular a methoxy group or a trifluoromethoxy group;
a C(O)—O(C1-C4) alkyl group, in particular a C(O)—O-methyl group;
a (C3-C6) cycloalkyl group, in particular cyclopropyl group;
a (C1-C4) alkylthio group, in particular a methylthio group; and
a pentafluorosulfanyl group;
F5 is selected from the group consisting of:
a halogen atom, in particular a fluorine atom; and
a (C1-C4) alkyl group unsubstituted or substituted with one or more fluorine atoms, in particular a trifluoromethyl group;
F6 is selected from the group consisting of a hydrogen atom and a halogen atom, in particular a fluorine atom;
F7 is independently selected from the group consisting of:
a halogen atom, in particular a fluorine atom;
a (C1-C4) alkyl group, in particular a methyl group;
a hydroxy group;
a (C1-C4) alkoxy group, in particular a methoxy group;
s is 0, 1 or 2;
and mixtures thereof.

2. The therapeutic combination according to claim 1, wherein the at least one KRAS G12C inhibitor is selected from the group consisting of:
compounds of formula (T) or a pharmaceutically acceptable salt thereof 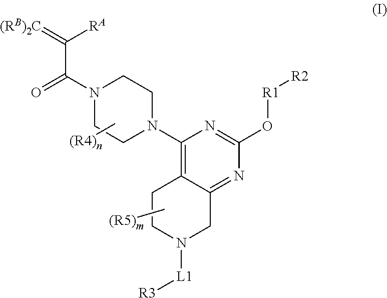
wherein
R^{A} is hydrogen, a (C1-C4) alkyl group or a halogen atom, in particular R^{A} is a fluorine atom;
R^{B} are independently of each other a hydrogen atom or a (C1-C4) alkyl group, in particular both R^{B} are hydrogen atoms;
R1 is a (C1-C3) alkylene group, in particular a methylene group;
R2 is a (C4-C8) heterocyclyl group comprising 1 or 2 heteroatoms selected from a nitrogen atom, an oxygen atom and a sulfur atom, in particular an oxygen atom and a nitrogen atom, and more particularly 1 or 2 nitrogen atom(s), such as a pyrrolidinyl group, unsubstituted or substituted with one or more R6;
L1 is a bond or a (C1-C3) alkylene group, in particular L1 is a bond;
R3 is a (C6-C10) aryl group or a heteroaryl group containing between 4 and 9 carbon atoms and 1 or 2 heteroatoms selected from a nitrogen atom, an oxygen atom and a sulfur atom, unsubstituted or substituted with one or more R7 groups; in particular R3 is a naphthyl group substituted with one or more R7 groups;
n is zero, 1 or 2, and R4 is a (C1-C4) alkyl group unsubstituted or substituted with one or more cyano groups or halogen atom(s);
m is zero, 1 or 2; and R5 is a (C1-C4) alkyl group unsubstituted or substituted with one or more halogen atoms;
R6 is a (C1-C4) alkyl group and more particularly a methyl group;
R7 is selected from the group consisting of a halogen atom, in particular a chlorine atom, and a (C1-C4) alkyl group unsubstituted or substituted with one or more halogen atoms;
compounds of formula (II) or a pharmaceutically acceptable salt thereof 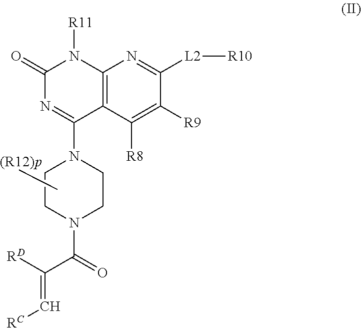
wherein:
R8 is a hydrogen atom, a halogen atom, for example a fluorine atom, or a (C1-C3) alkyl group unsubstituted or substituted with one or more halogen atoms, in particular with one or more fluorine atoms; in particular R8 is a hydrogen atom;
R9 is a halogen atom, in particular a fluorine atom or a (C1-C3) alkyl group unsubstituted or substituted with one or more halogen atoms, in particular with one or more fluorine atoms; in particular R9 is a fluorine atom;
L2 is a single bond or a methylene group; in particular a single bond;
R10 is a (C6-C10) aryl group or a heteroaryl group containing between 4 and 9 carbon atoms and 1 or 2 heteroatoms selected from a nitrogen atom, an oxygen atom and a sulfur atom, in particular a phenyl group, unsubstituted or substituted with one or more R13 groups;
R11 is a (C6-C10) aryl group or a heteroaryl group containing between 4 and 9 carbon atoms and 1 or 2 heteroatoms selected from a nitrogen atom, an oxygen atom and a sulfur atom, unsubstituted or substituted, preferably in ortho positions, with one or more R14 groups,
R^{C} is a hydrogen atom or a (C1-C3) alkyl group,
R^{D} is a hydrogen atom, a (C1-C3) alkyl group or a halogen atom in particular a fluorine atom;
p is zero, 1 or 2; and R12 is a (C1-C3) alkyl group, in particular a methyl group;
R13 is selected from the group consisting of a halogen atom, in particular a fluorine atom, a hydroxyl group (OH) and a (C1-C3) alkoxy group;
R14 is selected from the group consisting of a hydrogen atom, a (C1-C4) alkyl group and a (C3-C6) cycloalkyl group;
and mixtures thereof.

3. The therapeutic combination according to claim 2, wherein the at least one KRAS G12C inhibitor is a compound of formula (I′): 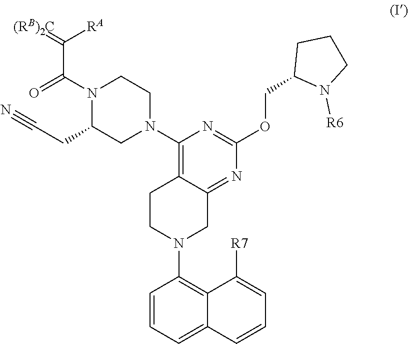
wherein:
R^{A} is a halogen atom, in particular a fluorine atom;
R^{B} are independently of each other hydrogen or a (C1-C4) alkyl; in particular both R^{B} are hydrogen atoms;
R6 is as defined in claim 2 and more particularly a methyl group; and
R7 is a halogen atom, in particular a chlorine atom;
or a pharmaceutically acceptable salt thereof.

4. The therapeutic combination according to claim 2, wherein the at least one KRAS G12C inhibitor is a compound of formula (II′): 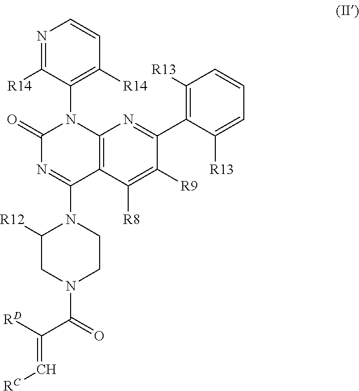
wherein:
R^{C} and R^{D} are as defined in claim 2, in particular both R^{C} and R^{D} are hydrogen atoms;
R8 is as defined in claim 2, in particular R8 is a hydrogen atom,
R9 is as defined in claim 2, in particular a halogen atom and more particularly a fluorine atom,
R12 is as defined in claim 2, in particular a methyl group,
R13 are as defined in claim 2, in particular one is a hydroxyl group and the other a fluorine atom,
R14 are as defined in claim 2, in particular are (C1-C3) alkyl groups; and more particularly one is a methyl group and the other an isopropyl group,
or a pharmaceutically acceptable salt thereof.

5. The therapeutic combination according to claim 1, wherein the at least one KRAS G12C inhibitor is:
2-((S)-4-(7-(8-chloronaphthalen-1-yl)-2-(((S)-1-methylpyrrolidin-2-yl) methoxy)-5,6,7,8-tetrahydropyrido[3,4-d]pyrimidin-4-yl)-1-(2-fluoroacryloyl) piperazin-2-yl) acetonitrile;
4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2 (1H)-one;
or a pharmaceutically acceptable salt thereof.

6. The therapeutic combination according to claim 1, wherein the at least one TEAD inhibitor is a compound of formula (IV), wherein:
q is 0,
G1 is a single bond or a methylene group,
G2 is a phenyl group unsubstituted or substituted with one or more G3 groups,
G3 a (C1-C4) alkyl group substituted with one or more fluorine atoms, in particular a trifluoromethyl group;
G4 is a hydrogen atom;
G7 is a hydrogen atom or a (C1-C4) alkyl group, especially a methyl group, unsubstituted or substituted with a (C1-C3) alkoxy group, especially a methoxy group;
G8 is a hydrogen atom;
G11 is a hydrogen atom;
or a pharmaceutically acceptable salt thereof.

7. The therapeutic combination according to claim 1, wherein the at least one TEAD inhibitor is a compound of formula (V′), 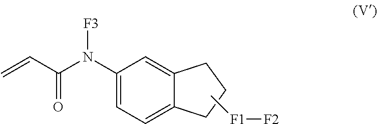
wherein:
F1 is selected from the group consisting of an oxygen atom and a group —N(H)—;
F2 is selected from the group consisting of:
a phenyl group unsubstituted or substituted with one or more F4 groups;
a benzyl group unsubstituted or substituted with one or more trifluoromethyl groups;
provided that F1 is NH;
a cyclohexyl group substituted with one or more F5 groups;
a pyridinyl group substituted with one or more F5 groups; and
a (C2-C4) alkyl group substituted with one or more fluorine atoms, in particular a (C2-C3) alkyl group substituted with a trifluoromethyl group;
F3 is selected from the group consisting of a hydrogen atom and a methyl group;
F4 is selected from the group consisting of a fluorine atom, a methyl group, a trifluoromethyl group, a methoxy group, a trifluoromethoxy group and a —C(O)—O-methyl group group; and
F5 is selected from the group consisting of a fluorine atom and a trifluoromethyl group;
or a pharmaceutically acceptable salt thereof.

8. The therapeutic combination according claim 1, wherein the at least one TEAD inhibitor is:
N-(3-(4-chlorophenoxy)-4-methylphenyl) acrylamide;
N-(1-(4-(trifluoromethyl)phenyl)-1H-indol-5-yl) acrylamide;
N-(3-(methoxymethyl)-1-(4-(trifluoromethyl)phenyl)-1H-indol-5-yl) acrylamide;
N-(3-methyl-1-(3-(trifluoromethyl)benzyl)-1H-indol-5-yl) acrylamide;
N-(1-((3-(trifluoromethyl)phenyl)amino)-2,3-dihydro-1H-inden-5-yl) acrylamide;
N-(3-(((trans)-4-(trifluoromethyl)cyclohexyl)oxy)-2,3-dihydro-1H-inden-5-yl) acrylamide;
N-(3-((3,4-difluorophenyl)amino)-2,3-dihydro-1H-inden-5-yl) acrylamide;
N-(3-(4-(trifluoromethyl) phenoxy)-2,3-dihydro-1H-inden-5-yl) acrylamide;
or a pharmaceutically acceptable salt thereof.

9. A pharmaceutical composition characterized in that it comprises a therapeutic combination as claimed according to claim 1 and at least one pharmaceutically acceptable excipient, diluent and/or carrier.

10. A therapeutic combination according to claim 1 for use in the treatment of KRAS G12C-mediated cancers.

11. The therapeutic combination for its use according to claim 10, in the treatment of lung adenocarcinoma, pancreatic ductal adenocarcinoma, rectum adenocarcinoma, colon adenocarcinoma, bile duct carcinoma, chronic myelomonocytic leukemia, rhabdomyosarcoma, endometrial cancer, bladder cancer, and ovarian cancer; and more particularly of non-small cell lung, small cell lung, pancreatic or colorectal cancer.

12. The therapeutic combination for its use according to claim 10, wherein said KRAS G12C inhibitor and said TEAD inhibitor are simultaneously administered in the same dosage form, simultaneously administered in separate dosage forms or separately administered.

13. A kit comprising in one or more separate packages a therapeutic combination as claimed in claim 1, optionally together with instructions for administration thereof and/or with a medical device for administration
